(19) United States
(12) Patent Application Publication
(10) Pub. No.: US 2007/0038294 A1
(43) Pub. Date: Feb. 15, 2007

(54) Method and apparatus for replacing a mitral valve and an aortic valve with a homograft

(76) Inventor: Jose L. Navia, Shaker Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/471,866

(22) Filed: Jun. 21, 2006

Related U.S. Application Data
(63) Continuation-in-part of application No. 11/037,499, filed on Jan. 18, 2005.
(60) Provisional application No. 60/537,838, filed on Jan. 21, 2004.
(60) Provisional application No. 60/795,259, filed on Apr. 26, 2006.

(51) Int. Cl. A61F 2/24

(52) U.S. Cl. 623/2.13; 623/1.26

(57) ABSTRACT

A stentless bioprosthetic graft for repairing a first native heart valve and a second native heart valve in a heart. The bioprosthetic graft includes a harvested homograft having a harvested mitral valve portion, a harvested distal aorta, and an extension portion made of a biocompatible material. The harvested distal aorta further includes a harvested aortic valve, a harvested aortic root, and at least a portion of a harvested atrial wall. The harvested mitral valve portion is for suturing place of the first native heart valve, and the harvested distal aorta is for suturing to a partial section of the second native heart valve. The extension portion is sutured to the homograft and is for suturing to the left atrial wall of the heart to close an incision in the left atrial wall following implantation of the harvested mitral valve portion and the harvested distal aorta of the homograft.

### RELATED APPLICATIONS

This application is a continuation-in-part of U.S. patent application Ser. No. 11/037,499, filed Jan. 18, 2005, which claims priority from U.S. Provisional Patent Application No. 60/537,838, filed Jan. 21, 2004, which is herein incorporated by reference in its entirety. This application also claims priority from U.S. Provisional Patent Application No. 60/795,259, filed Apr. 26, 2006, which is herein incorporated by reference in its entirety.

### TECHNICAL FIELD

The present invention relates to an apparatus and methods for treating diseased heart valves, and is particularly directed to an apparatus and methods for repairing both a mitral valve and an aortic valve with a homograft.

### BACKGROUND OF THE INVENTION

It is known to replace a diseased mitral valve with a stented or unstented bioprosthetic valve. The bioprosthetic mitral valve can be made from a harvested biological tissue including bovine, equine or porcine pericardial tissue, a bovine, equine or porcine mitral valve, or a homograft (or allograft) mitral valve. The bioprosthetic mitral valve can also be made from a suitable synthetic material including such as polyurethane, expanded PTFE, or Gore-Tex®.

It is also known to replace a diseased aortic valve with a stented or unstented bioprosthetic valve. The bioprosthetic aortic valve can be made from a harvested biological tissue including bovine, equine or porcine pericardial tissue, bovine, equine or porcine aortic valve, or a homograft (or allograft) aortic valve. The bioprosthetic aortic valve can also be made from a suitable synthetic material such as polyurethane, expanded PTFE, or Gore-Tex®.

In some cases, both the mitral valve and the aortic valve are diseased and a need therefore exists for a method and apparatus for replacing both valves with a bioprosthetic implantation in a single procedure. The present invention addresses this need using a stentless homograft.

### SUMMARY OF THE INVENTION

In accordance with one aspect of the present invention, a stentless bioprosthetic graft is provided for repairing a first native heart valve and a second native heart valve in a heart. The bioprosthetic graft includes a harvested homograft having a harvested mitral valve portion, a harvested distal aorta, and an extension portion made of a biocompatible material. The harvested distal aorta further includes a harvested aortic valve, a harvested aortic root, and at least a portion of a harvested aortic wall. The harvested mitral valve portion is for suturing in place of the first native heart valve, and the harvested distal aorta is for suturing to a partial section of the second native heart valve. The extension portion is sutured to the homograft, and is for suturing to the left atrial wall of the heart to close an incision in the left atrial wall following implantation of the harvested mitral valve portion and the harvested distal aorta of the homograft.

In accordance with another aspect of the present invention, a method is provided for repairing a first native heart valve and a second native heart valve in a heart with a bioprosthetic graft. A homograft that includes both a harvested mitral valve portion and a harvested distal aorta is first harvested. The distal aorta includes a harvested aortic root, a harvested aortic valve, and at least a portion of a harvested atrial wall. The harvested mitral valve portion is for suturing in place of the first native heart valve, and the harvested distal aorta is for suturing to a partial section of the second native heart valve. The majority of the anterior and posterior leaflets of the first native heart valve are then resected from the valve annulus while leaving the free edges of the anterior and posterior leaflets intact along with the native chordae tendinea so that the native chordae tendinea can provide prolapse prevention and left ventricular muscle support for the harvested mitral valve portion of the homograft in addition to maintaining the continuity between the valve annulus and the papillary muscles. A biocompatible extension portion is then sutured to the homograft. Next, first and second leaflets of the harvested mitral valve portion are sutured to the free edges of the anterior and posterior leaflets of the first native heart valve that remain following resection of the first native heart valve. The annulus of the harvested mitral valve portion of the homograft is then sutured to the annulus of the first native heart valve to secure the bioprosthetic graft to the valve annulus. Lastly, the harvested distal aorta of the homograft is sutured to the partial section of the second native heart valve, and the extension portion is sutured to the left atrial wall of the heart to close the left atrial wall.

In accordance with another aspect of the present invention, a stentless bioprosthetic graft for repairing both a first native heart valve and a second native heart valve is provided. The bioprosthetic graft includes a harvested homograft having at least a portion of a harvested mitral valve and at least a portion of a harvested distal aorta. The harvested distal aorta includes a harvested aortic root, a harvested aortic valve, and at least a portion of a harvested atrial wall. The at least a portion of a harvested mitral valve is for suturing to a partial section of the first native heart valve, and the at least a portion of a harvested distal aorta is for suturing to a partial section of the second native heart valve. The at least a portion of a harvested atrial wall is for suturing to the native atrial wall of the heart to close the native atrial wall following implantation of the harvested homograft.

In accordance with another aspect of the present invention, a method is provided for repairing both a first native heart valve and a second native heart valve of a heart with a bioprosthetic graft. The first native heart valve has at least one leaflet connected to an annulus. The at least one leaflet has a free edge and is further connected to papillary muscles by chordae tendinea. A homograft is harvested that includes at least a portion of a harvested mitral valve and at least a portion of a harvested distal aorta. The distal aorta includes a harvested aortic root, a harvested aortic valve, and at least a portion of a harvested atrial wall. The at least a portion of a harvested mitral valve is for suturing to a partial section of the first native heart valve, and the at least a portion of a harvested distal aorta is for suturing to a partial section of the second native heart valve. Next, at least a portion of the first and second native heart valves is resected from the heart, and the at least a portion of a harvested mitral valve is sutured in place of the first native heart valve. The at least a portion of a harvested distal aorta is sutured in place of the second native heart valve, and the at least a portion of a harvested atrial wall is sutured to the native atrial wall of the heart to close the native atrial wall.

In accordance with another aspect of the present invention, a stentless bioprosthetic graft for repairing a native tricuspid valve of a heart is provided. The bioprosthetic graft includes a harvested homograft having at least a portion of a harvested mitral valve. The least a portion of a harvested mitral valve is for suturing to a partial section of the native tricuspid valve.

In accordance with another aspect of the present invention, a method is provided for repairing a native tricuspid valve of a heart with a bioprosthetic graft. The native tricuspid valve has at least one leaflet connected to an annulus. The at least one leaflet has a free edge and is further connected to papillary muscles by chordae tendinea. A homograft that includes at least a portion of a harvested mitral valve is first harvested. The at least a portion of a harvested mitral valve is for suturing to a partial section of the native tricuspid valve. Next, at least a portion of the native tricuspid valve is resected from the heart, and the at least a portion of a harvested mitral valve is sutured to the partial section of the native tricuspid valve.

In accordance with another aspect of the present invention, a stentless bioprosthetic graft for repairing a native tricuspid valve of a heart is provided. The bioprosthetic graft includes a harvested homograft having a harvested mitral valve. The harvested mitral valve is for suturing in place of the native tricuspid valve.

In accordance with another aspect of the present invention, a method is provided for repairing a native tricuspid valve of a heart with a bioprosthetic graft. The native tricuspid valve has at least one leaflet connected to an annulus, and the at least one leaflet has a free edge further connected to papillary muscles by chordae tendinea. A homograft that includes a harvested mitral valve and is for suturing in place of the native tricuspid valve is first harvested. Next, the native tricuspid valve is resected from the heart, and the harvested mitral valve is sutured in place of the native tricuspid valve.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view of a stentless bioprosthetic graft for implantation into a recipient heart constructed in accordance with the present invention;

FIG. 2 is a plan view showing the bioprosthetic graft of FIG. 1;

FIG. 3 is a perspective view showing another alternative embodiment of bioprosthetic graft shown in FIG. 1;

FIG. 4 is a perspective view of the recipient heart showing an incision in preparation for excision of the native mitral and aortic valves;

FIG. 5 is a perspective view of the recipient heart with the diseased native mitral and aortic valves exposed for excision;

FIG. 6 is a perspective view of the left ventricle of the recipient heart illustrating the native mitral valve being resected;

FIG. 7 is a plan view of FIG. 6 following resection of the native mitral valve;

FIG. 8 is a perspective view of the recipient heart following excision of the diseased native mitral and aortic valves showing the free edges of the preserved native mitral valve leaflets and the native chordae tendinea;

FIG. 9 is a perspective view showing the bioprosthetic graft of FIG. 1 being sutured to the free edges of the native anterior mitral valve leaflets;

FIG. 10 is a perspective view showing the bioprosthetic graft of FIG. 1 being sutured to the mitral valve annulus;

FIG. 11 is a perspective view showing the bioprosthetic graft shown in FIG. 1 implanted in the recipient heart;

FIG. 12 is a plan view of FIG. 11 following implantation of the bioprosthetic graft;

FIG. 13 is a perspective view showing the bioprosthetic graft of FIG. 1 being sutured in place;

FIG. 14 is a perspective view showing the bioprosthetic graft of FIG. 1 being sutured to the coronary sinuses of the recipient heart and the left atrium being closed with an extension portion of the bioprosthetic graft;

FIG. 15 is a perspective view of the recipient heart following implantation of the bioprosthetic graft shown in FIG. 1;

FIG. 16 is an alternate embodiment of the bioprosthetic graft shown in FIG. 1;

FIG. 17A is a plan view showing the bioprosthetic graft of FIG. 16;

FIG. 17B is a plan view showing an alternative embodiment of the bioprosthetic graft shown in FIG. 16;

FIG. 17C is a plan view showing another alternative embodiment of the bioprosthetic graft shown in FIG. 16;

FIG. 17D is a plan view showing another alternative embodiment of the bioprosthetic graft shown in FIG. 16;

FIG. 18 is a perspective view showing another alternative embodiment of the bioprosthetic graft shown in FIG. 16;

FIG. 19 is a perspective view of the recipient heart showing an incision in preparation for excision of the native mitral and aortic valves;

FIG. 20 is a perspective view of the recipient heart with the diseased native mitral and aortic valves exposed for excision;

FIG. 21 is a perspective view of the recipient heart following excision of the diseased native mitral and aortic valves showing the free edges of the preserved native mitral valve leaflets and the native chordae tendinea;

FIG. 22 is a perspective view of the left ventricle of the recipient heart illustrating a portion of the native mitral valve being resected;

FIG. 23 is a plan view of FIG. 22 following resection of the portion of the native mitral valve;

FIG. 24 is a perspective view showing the bioprosthetic graft of FIG. 16 being sutured to the free edges of the native anterior mitral valve leaflets;

FIG. 25 is a perspective view showing the bioprosthetic graft of FIG. 16 being sutured to the mitral valve annulus;

FIG. 26 is a perspective view showing a partial portion of the bioprosthetic graft shown in FIG. 16 implanted in the recipient heart;

FIG. 27 is a perspective view showing the bioprosthetic graft of FIG. 16 being sutured to the coronary sinuses of the recipient heart and the left atrium being closed with an at least a portion of a harvested atrial wall of the homograft;

FIG. 28 is a perspective view of the recipient heart following implantation of the bioprosthetic graft shown in FIG. 16;

FIG. 29 is a perspective view showing another alternative embodiment of a stentless bioprosthetic graft for implantation into a recipient heart constructed in accordance with the present invention;

FIG. 30 is a perspective view showing an alternative embodiment of the bioprosthetic graft in FIG. 29;

FIG. 31A is a perspective view showing an alternative embodiment of the bioprosthetic graft in FIG. 30;

FIG. 31B is a perspective view showing another alternative embodiment of the bioprosthetic graft in FIG. 30;

FIG. 31C is a perspective view showing another alternative embodiment of the bioprosthetic graft in FIG. 30; and

FIG. 32 is a perspective view of a recipient heart with a diseased native tricuspid valve exposed for excision.

### DESCRIPTION OF EMBODIMENTS

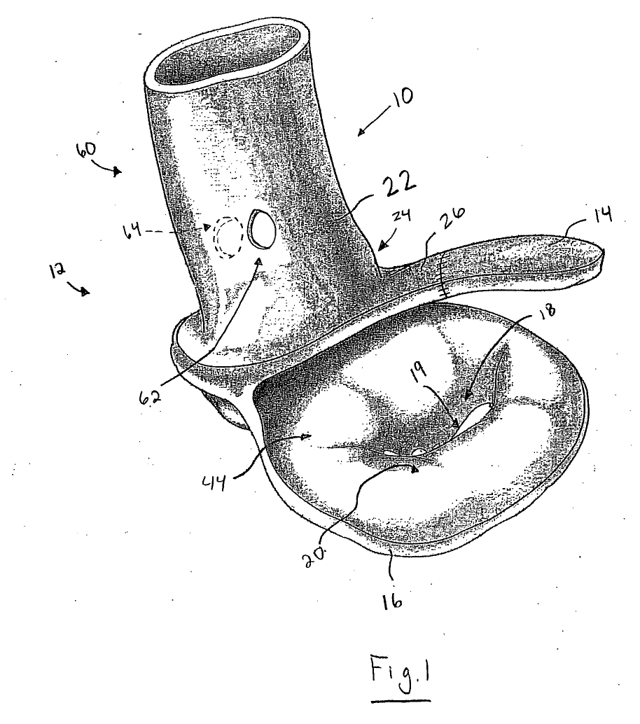

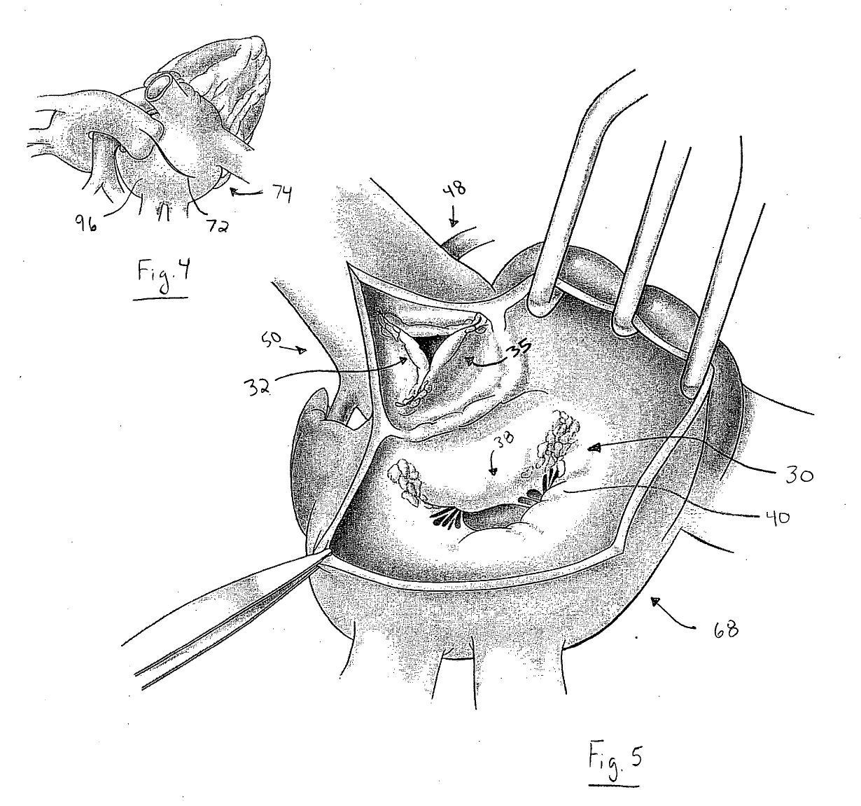

The present invention relates to an apparatus and methods for treating diseased heart valves, and is particularly directed to an apparatus and methods for repairing both a mitral valve and an aortic valve with a homograft. As representative of the present invention, FIG. 1 illustrates a stentless bioprosthetic graft 10 for repairing both a first native heart valve 30 (FIG. 5), such as a mitral valve 34, and a second native heart valve 32, such as an aortic valve 35.

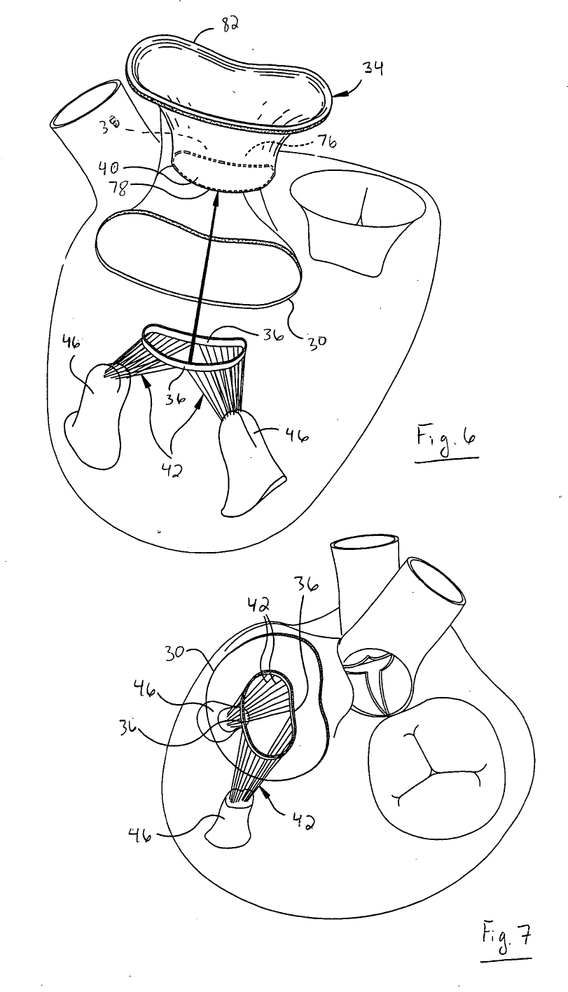

As shown in FIG. 1, the bioprosthetic graft 10 comprises a harvested homograft 12 and an extension portion 14. The homograft 12 includes a harvested mitral valve portion 16 that can be sutured in place of a native mitral valve 34 (FIG. 5). The harvested mitral valve portion 16 (FIG. 1) includes first and second leaflets 18 and 20 for suturing to the free edges 36 of the anterior and posterior leaflets 38 and 40 (FIG. 5), respectively, of the native mitral valve 34. As described in more detail below, the anterior and posterior leaflets 38 and 40 of the native mitral valve 34 may be left intact following resection of the native mitral valve so that the native chordae tendinea 42 (FIG. 6) continue to provide prolapse prevention and left ventricular muscle support, in addition to maintaining the continuity between the harvested mitral valve annulus 44 (FIG. 1) and the papillary muscles 46 (FIG. 6).

The homograft 12 further includes a harvested distal aorta 22 that can be sutured to a partial section 48 (FIG. 5) of the second native heart valve 32, such as a native aortic root 50. More particularly, the partial section 48 may comprise a supracoronary portion (not shown) or a subcoronary portion 52 (FIG. 8) of the native aortic root 50 (FIG. 5), depending upon the manner in which the native distal aorta 54 is resected from a donor heart (not shown). As shown in FIG. 8, for example, the partial section 48 comprises a subcoronary portion 52 as the native distal aorta 54 has been transected below the native left and right coronary sinuses 56 and 58.

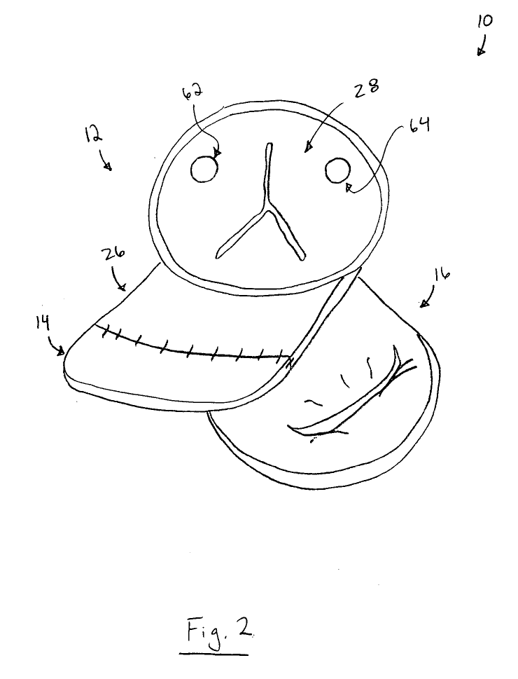

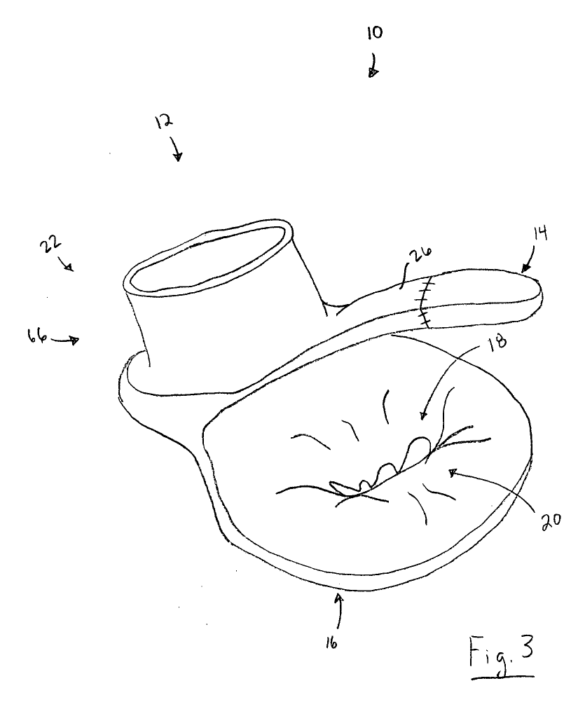

The harvested distal aorta 22 (FIG. 1) further includes a harvested aortic root 24, at least a portion of a harvested atrial wall 26, and a harvested aortic valve 28 (FIG. 2). As shown in FIGS. 1 and 3, the harvested aortic root 24 can have a variety of different configurations, depending upon the manner in which the harvested distal aorta 22 is transected from the donor heart. Referring to FIG. 1, for example, the harvested distal aorta 22 may be transected so that the harvested aortic root 24 includes a supracoronary portion 60 having left and right coronary sinuses 62 and 64. Alternatively, the harvested distal aorta 22 may be transected below the coronary sinuses 62 and 64 so that the harvested aortic root 24 has a subcoronary portion 66 not including the right and left coronary sinuses (FIG. 3).

The extension portion 14 of the bioprosthetic graft 10 is for suturing to an atrial wall 96 (FIG. 5) of a native or recipient heart 68 to close the atrial wall following implantation of the bioprosthetic graft. The extension portion 14 may be made of a biocompatible material, such as autologous or heterologous pericardium (bovine, equine, porcine, etc.) or other biological tissue. Alternatively, the extension portion 14 may be made of a biocompatible artificial tissue, such as polyurethane, PTFE or Gore-Tex® (W. L. Gore & Associates, Inc., Flagstaff, Ariz.). It should be appreciated that the bioprosthetic graft 10 can by cryopreserved and/or tanned (fixed) as known in the art following harvest.

Prior to implantation into the recipient heart 68, the homograft 12 shown in FIG. 1 may be harvested as described below. First, the left atrium (not shown) of a donor heart (not shown) is opened in a manner such that the dome (not shown) at the level of the aortic root (not shown) is preserved. The mitral annulus (not shown), leaflets (not shown), chordae tendinae (not shown) and papillary muscles (not shown) of the donor heart are then anatomically evaluated. Next, the height of the anterior and posterior mitral leaflets is measured by taking either direct or echocardiographic measurements, for example. The distal ascending aorta (not shown) is then transected proximal to the right and left coronary sinuses (not shown). Thereafter, the harvested distal aorta 22 is anatomically evaluated and measured.

Next, the left ventricle (not shown) of the donor heart is opened below the papillary muscle level (not shown). An incision is placed near the donor mitral valve (not shown) and around the posterior area (not shown) of the mitral annulus, taking care to preserve the aortic-mitral membrane (not shown), the ascending aorta (not shown), the dome of the left atrium at the reflection of the aortic root, and the mitral valve annulus. Doing so ensures that these components of the homograft 12 remain intact as a single unit.

The entire mitral valve of the donor heart is then excised or removed by incision of the valve circumferentially. The chordae tendinea that remain attached to the valve leaflets are then removed along with the tips of the corresponding papillary muscles. Alternatively, the chordae tendinea and corresponding tips of the papillary muscles may be preserved. The homograft 12 can then be frozen or otherwise preserved for implantation.

After the homograft 12 is thawed for implantation, any excess myocardium may be trimmed as needed. For example, excess myocardium of the atrial wall 96 and/or the left ventricle (not shown) may be cut away from the harvested mitral annulus 44 and aortic valve annulus (not shown) without damaging the leaflets 38 and 40, so that just enough tissue remains to allow sewing of the homograft 12 at the aortic root junction level 70 (FIG. 5). If needed, the chordae tendinea and the corresponding papillary muscles may be further trimmed from the free edges of the leaflets. Alternatively, the chordae tendinea and the corresponding papillary muscles may be preserved in a standard fashion for attachment to the left ventricular wall as disclosed in U.S. Pat. No. 6,074,417 (“the '417 patent”), the subject matter of which is herein incorporated by reference.

Based on direct or echocardiographic measurements of the native aortic valve 35 (FIG. 5), the native mitral valve 34, the height of the native mitral leaflets 38 and 40, the chordae tendinea 42 (FIG. 6), and the corresponding papillary muscles 46, an appropriately-sized homograft 12 (FIG. 1) is then chosen for implantation into the recipient heart 68. As shown in FIG. 4, an incision 72 is first made to open the left atrium 74 of the recipient heart 68. The incision 72 allows exposure of the native mitral valve 34 and native aortic valve 35 for excision as shown in FIG. 5.

Next, the distal aorta of the donor heart is resected so that the harvested distal aorta 22 includes the aortic root 24, the aortic valve 28, and at least a portion of an atrial wall 26 (e.g., a left atrial wall). The native aortic root 50 may be resected so that only a subcoronary portion 52 (FIG. 8) remains. Alternatively, the native aortic root 50 may be resected so that only a supracoronary portion (not shown) remains. After resection, the at least a portion of the atrial wall 26 is extended by attaching the extension portion 14.

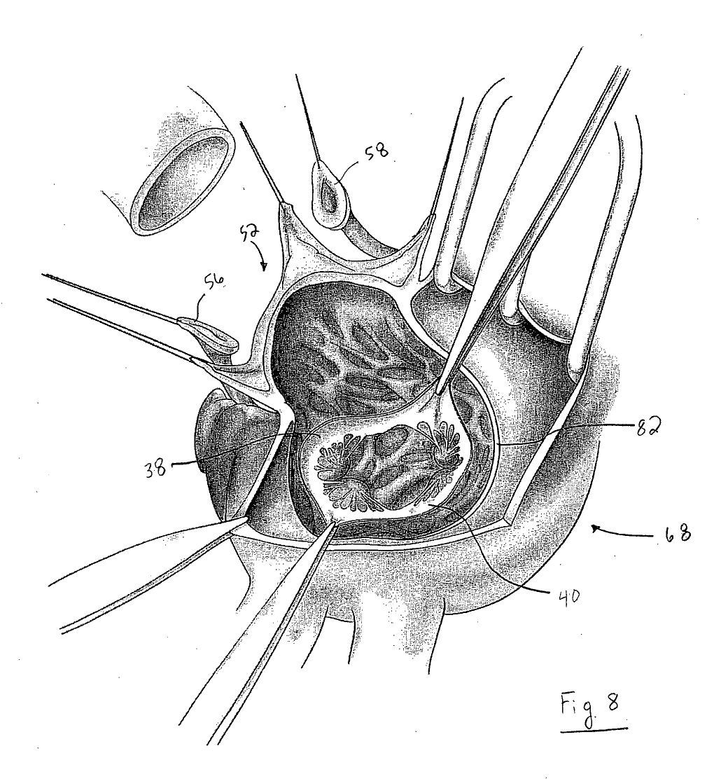

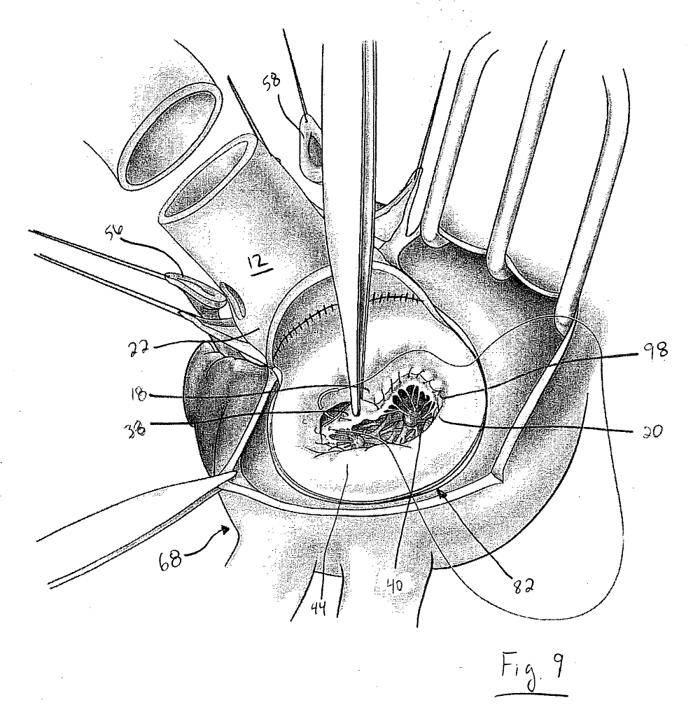

As illustrated in FIGS. 6 and 7, the native mitral valve 34 is then dissected from the recipient heart 68. Proximal and distal ends 76 and 78 of the native mitral valve annulus 82, which respectively include the anterior and posterior leaflets 38 and 40, are then resected so that the free edges 36 of the leaflets remain intact and connected to the native chordae tendinea 42 which, in turn, remain attached to the corresponding papillary muscles 46. It is important that the rough zone chordae tendinea (not show in detail) and the free edges 36 of the leaflets 38 and 40 are preserved.

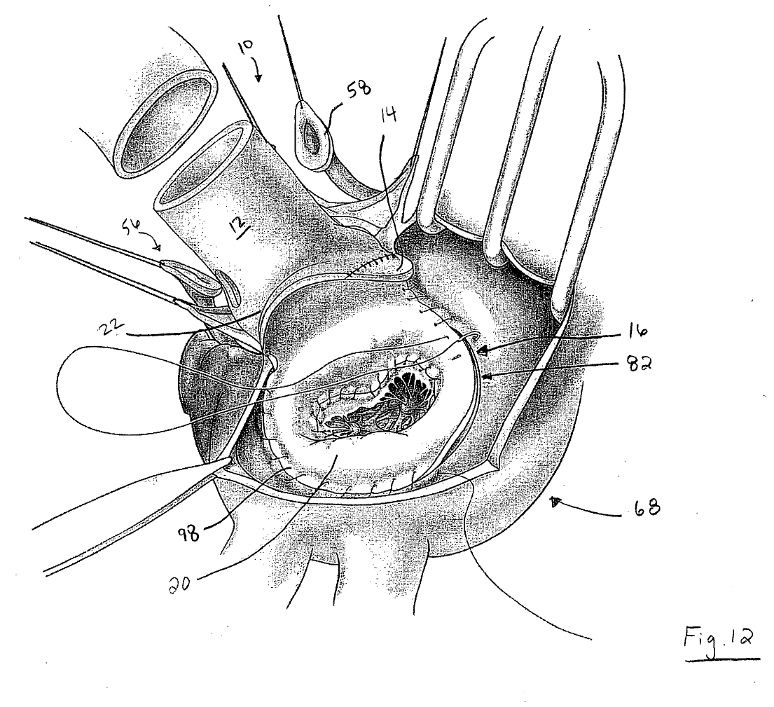

The homograft 12 is next moved into position for implantation. As shown in FIG. 9, the free edge 19 of the first leaflet 18 of the harvested mitral valve portion 16 is sutured down to the native anterior leaflet 38 with 5-0 Ethibond, Gore-Tex® or polypropylene (Prolene 5-0) continuous over-and-over sutures 98. This suture 98 may be started from the apex (or middle) of the first leaflet 18 and then toward both mitral commissures 80 (FIG. 11), which makes it easy to suture the homograft 12 to the native anterior leaflet 38. The same procedure is repeated for the attachment of the homograft 12 to the native posterior leaflet 40, except that the second leaflet 20 of the mitral valve portion 16 is sutured to the posterior leaflet of the native mitral valve 34. During this procedure, the rough zone chordae tendinea, the free edge (not shown) of the native posterior leaflet 40, and the cleft chordae tendinea (not shown), except for the basal chordae (not shown), must be preserved.

The annulus 44 of the harvested mitral valve portion 16 of the homograft 12 is then sutured down to the native mitral annulus 82 using continuous or interrupted stitches of 3-0 or 4-0 polypropylene or Ethibond sutures 98 as shown in FIG. 12. The fibrous trigones (not shown) of the homograft 12 are lined up with the fibrous trigones (not shown) of the homograft recipient. Attention is given to distributing the leaflet tissue of the homograft 12 uniformly around the native mitral annulus 82.

Additionally or optionally, the harvested mitral valve portion 16 of the homograft 12 can be supported by remodeling annuloplasty using an appropriately-sized partial or complete known annuloplasty mitral ring (not shown) that is sized to match the first leaflet 18 of the homograft, for example. The partial annuloplasty ring may be secured with sutures placed around the posterior inter-trigonal perimeter of the native mitral annulus 82. The complete annuloplasty ring may be secured with sutures placed around the entire perimeter of the native mitral annulus 82

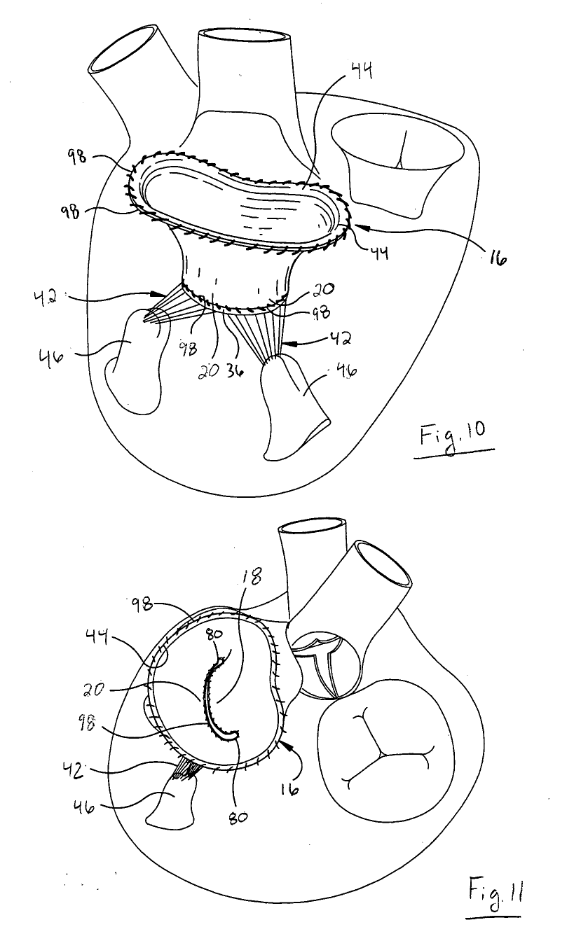

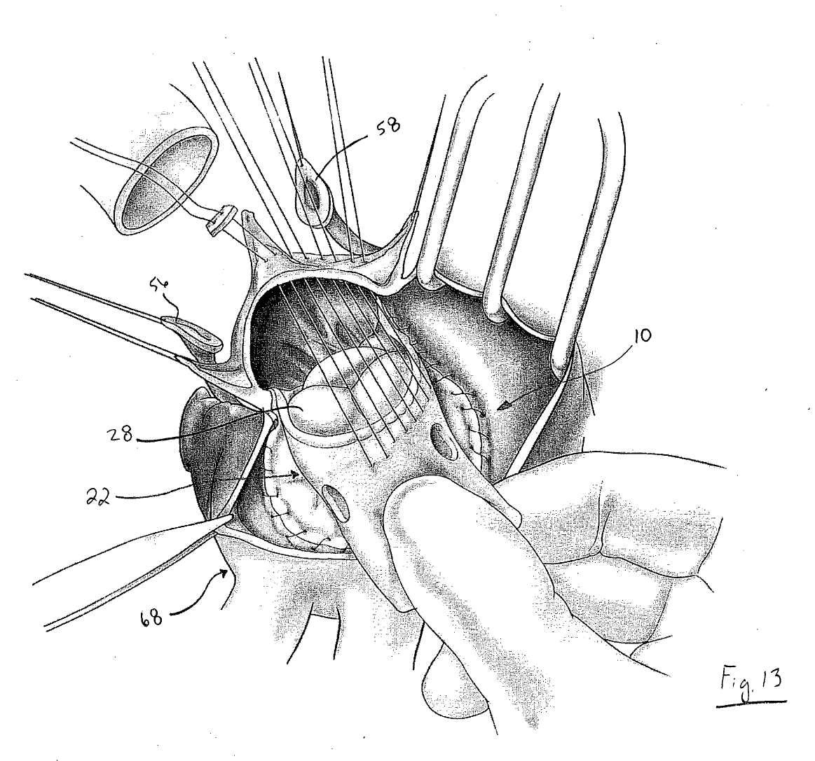

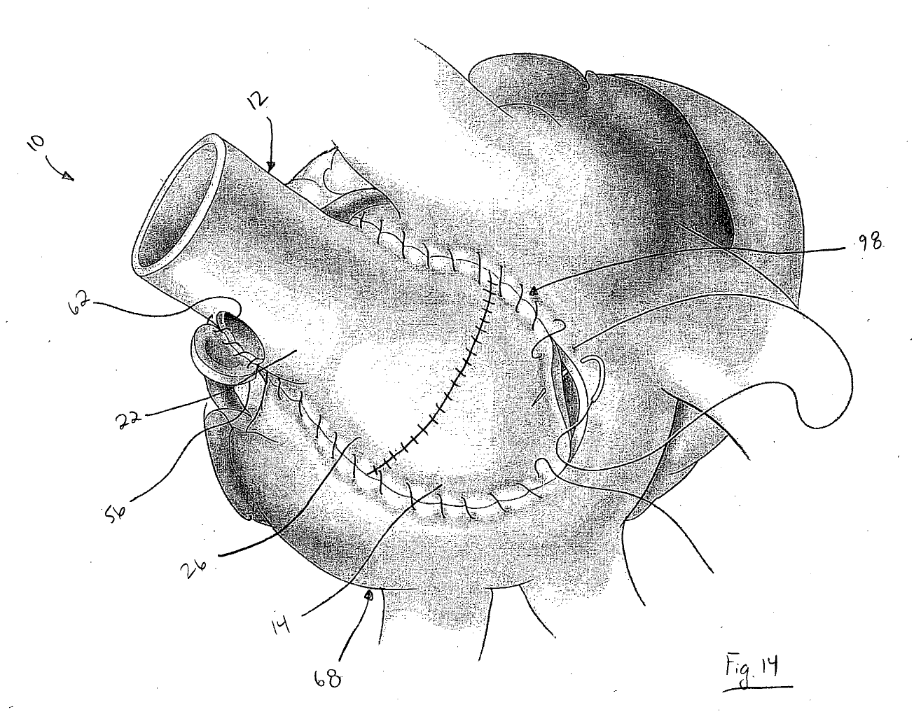

After the harvested mitral valve portion 16 is secured in the recipient heart 68 as shown in FIGS. 10 and 11, the harvested distal aorta 22 of the homograft 12 is then implanted in a known manner using a procedure referred to as a “Mini Root Technique”. As shown in FIGS. 13 and 14, the native right and left coronary sinuses 56 and 58 are attached to the harvested aortic root 24 of the homograft 12 with 4-0 or 5-0 polypropylene or Gore-Tex® sutures 98, and the left atrium 74 of the recipient heart 68 is closed using the extension portion 14 of the homograft. An exterior view of the recipient heart 68 following implantation of the homograft 12 is shown in FIG. 15.

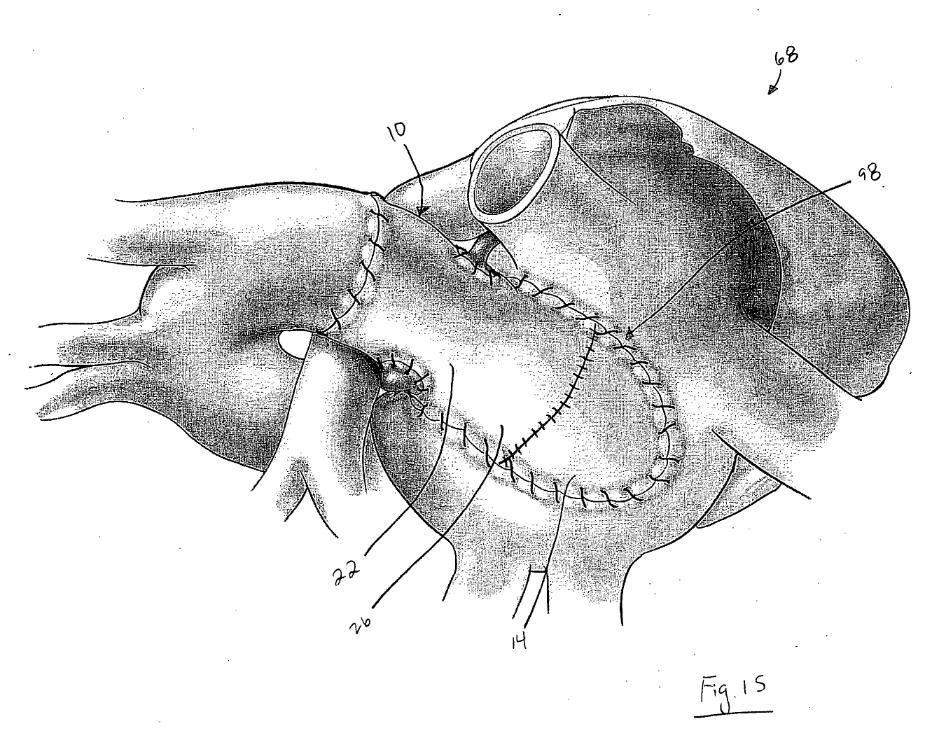

FIGS. 16-28 illustrate an alternative embodiment of the present invention. The bioprosthetic graft 10ₐ of FIGS. 16-28 is identically constructed as the bioprosthetic graft 10 of FIGS. 1-15, except where as described below. In FIGS. 16-28, structures that are identical as structures in FIGS. 1-15 use the same reference numbers, whereas structures that are similar but not identical carry the suffix “a”.

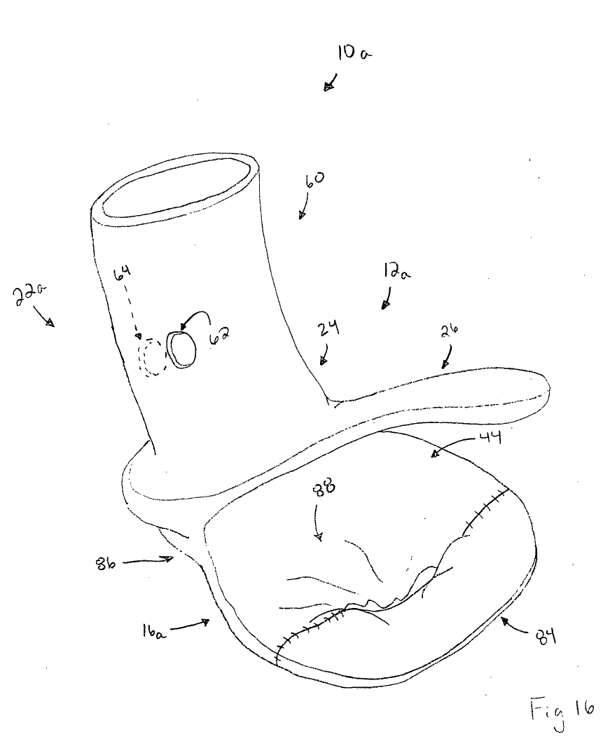

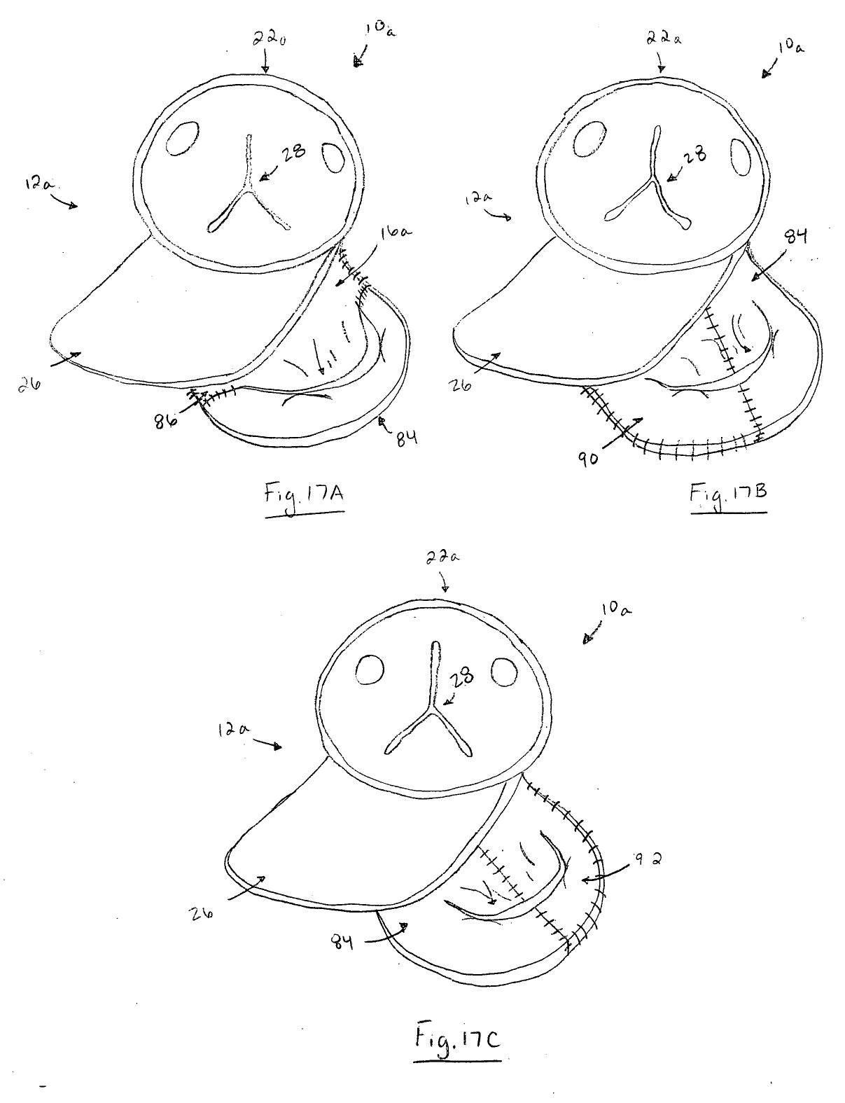

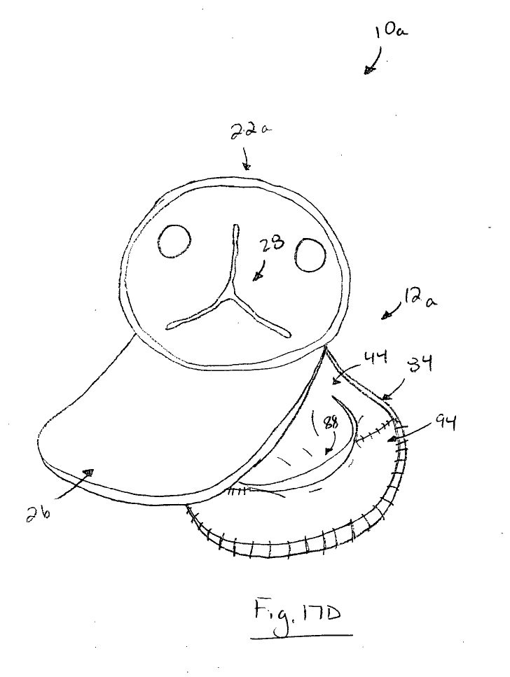

As shown in FIG. 16, the bioprosthetic graft 10ₐ comprises a harvested homograft 12ₐ. The homograft 12ₐ includes at least a portion 16ₐ of a harvested mitral valve that can be sutured to a partial section 84 of a first native heart valve 30, such as a native mitral valve 34 (FIG. 20). The at least a portion 16ₐ (FIG. 16) of a harvested mitral valve can include an anterior mitral region 86 comprising an anterior leaflet 88 as shown in FIGS. 16 and 17A. Alternatively, the at least a portion 16ₐ of a harvested mitral valve can also include an anterolateral portion 90 (FIG. 17B), a posteromedial portion 92 (FIG. 17C), or a posterior mitral region 94 (FIG. 17D).

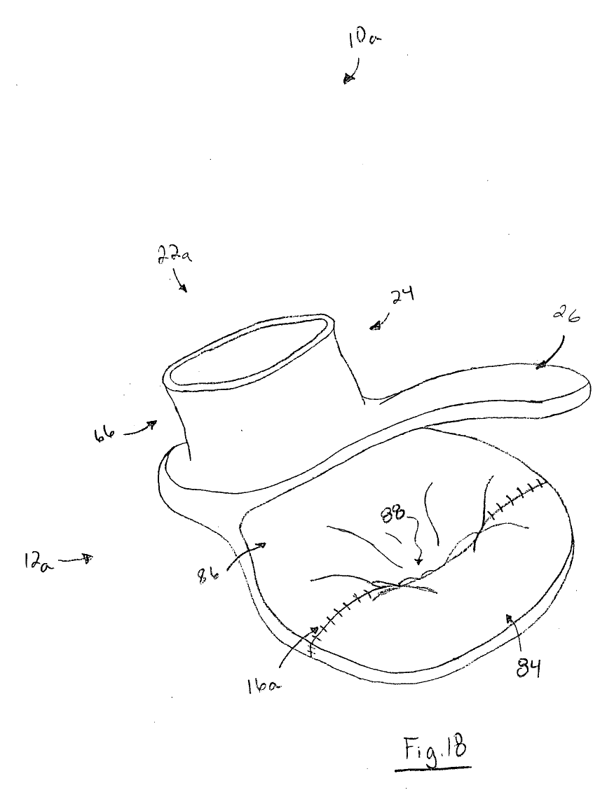

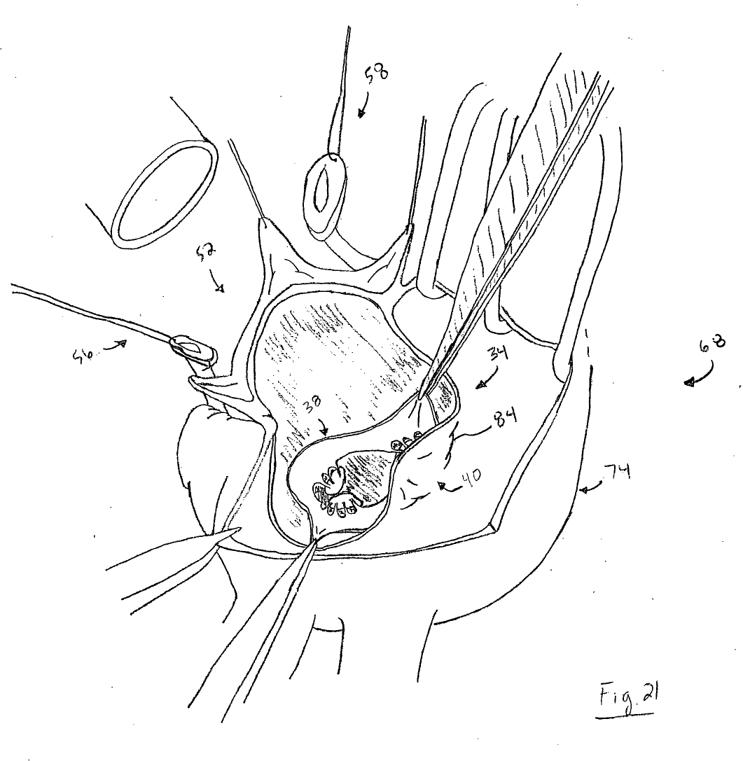

The homograft 12ₐ further includes at least a portion 22ₐ (FIG. 16) of a harvested distal aorta that can be sutured to a partial section 48 (FIG. 20) of a second native valve 32, such as a native aortic root 50. More particularly, the partial section 48 may comprise a supracoronary portion (not shown) or subcoronary portion 52 (FIG. 21) of the native aortic root 50 (FIG. 20), depending upon the manner in which the native distal aorta 54 is resected from a donor heart (not shown). As shown in FIG. 18, for example, the partial section 48 comprises a subcorornay portion 52 as the native distal aorta 54 has been transected below the native left and right coronary sinuses 56 and 58.

The at least a portion 22ₐ (FIG. 16) of a harvested distal aorta includes a harvested aortic root 24, a harvested aortic valve 28 (FIG. 17A), and at least a portion 26 (FIG. 16) of a harvested atrial wall. As shown in FIGS. 16 and 18, the harvested aortic root 24 can have a variety of different configurations, depending upon the manner in which the at least a portion 22ₐ of a harvested distal aorta is transected from the donor heart. Referring to FIG. 16, for example, the at least a portion 22ₐ of a harvested distal aorta may be transected so that the harvested aortic root 24 includes a supracoronary portion 60 having left and right coronary sinuses 62 and 64. Alternatively, the at least a portion 22ₐ of a harvested distal aorta may be transected below the coronary sinuses 56 and 58 so that the harvested aortic root 24 has a subcoronary portion 66 that does not include the right and left coronary sinuses (FIG. 18).

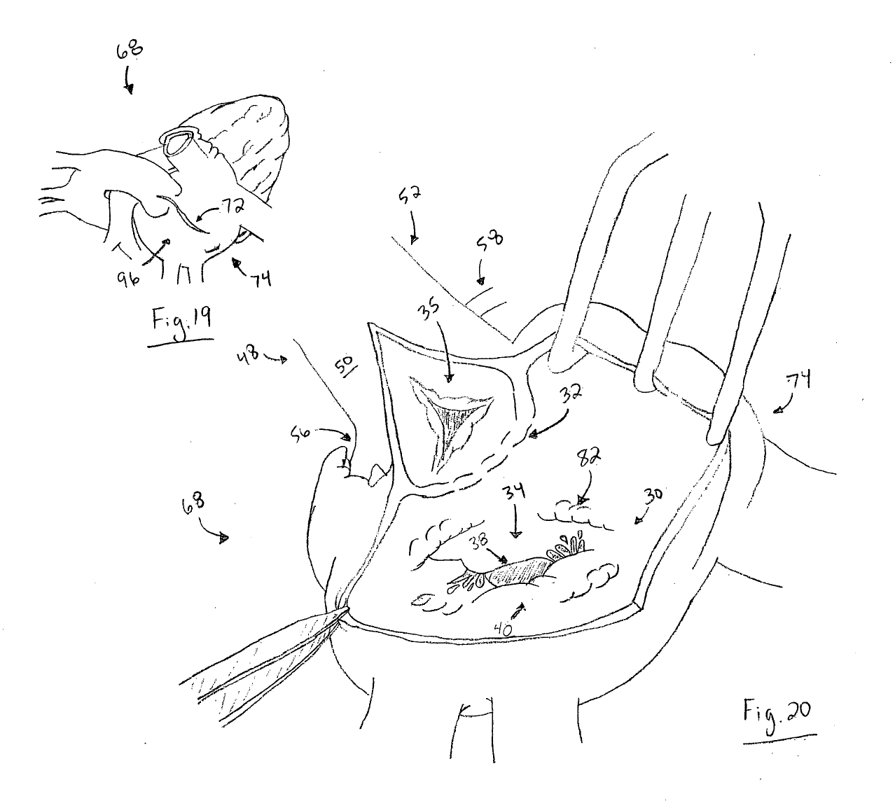

The at least a portion 22ₐ of a harvested distal aorta is for suturing to a partial section 48 of the second native heart valve 32. As will be described in more detail below, the harvested aortic root 24 of the at least a portion 22ₐ of a harvested distal aorta may be sutured to the native aortic root 50, and the at least a portion 26 of a harvested atrial wall is for suturing to the native atrial wall 96 of a recipient heart 68 (FIG. 19) to close the native atrial wall following implantation of the homograft 12ₐ.

Prior to implantation, the homograft 12ₐ shown in FIG. 16 may be harvested as described below. First, the left atrium (not shown) of a donor heart (not shown) is opened in a manner such that the dome (not shown) at the level of the aortic root (not shown) is preserved. The mitral annulus (not shown), posterior mitral leaflet (not shown), chordae tendinea (not shown) and papillary muscles (not shown) of the donor heart are then anatomically evaluated. Next, the height of the posterior mitral valve leaflet is measured by taking either direct or echocardiographic measurements, for example. The distal ascending aorta (not shown) is then transected either proximal to, or distal from, the two coronary sinuses (not shown). Thereafter, the at least a portion 22ₐ of a harvested distal aorta is anatomically evaluated and measured.

Next, the left ventricle (not shown) of the donor heart is opened below the papillary muscle level (not shown). An incision is placed near the donor mitral valve (not shown) and around the posterior area (not shown) of the mitral annulus, taking care to preserve the aortic-mitral membrane (not shown), the ascending aorta (not shown), the dome (not shown) of the left atrium at the reflection of the aortic root (not shown), and the mitral valve annulus. Doing so ensures that these components of the homograft 12ₐ remain intact as a single unit.

A portion of the native mitral valve 34 of the recipient heart 68 (FIG. 19) is then excised or removed by incision. Where, for example, the native anterior leaflet 38, but not the posterior leaflet 40, of the native mitral valve 34 is diseased, the donor mitral valve may be excised so that the at least a portion 16ₐ (FIG. 16) of a harvested mitral valve only includes the anterior leaflet 88 and surrounding harvested mitral annulus 44. The donor chordae tendinea that remain attached to the anterior mitral valve leaflet are then removed along with the tip of the corresponding papillary muscles. Alternatively, the chordae tendinea and corresponding tip of the papillary muscles may be preserved. The homograft 12ₐ can then be frozen or otherwise preserved for implantation.

After the homograft 122ₐ is thawed for implantation, any excess myocardium may be trimmed as needed. For example, excess myocardium of the atrial wall 96 and/or the left ventricle (not shown in detail) may be cut away from the harvested mitral annulus 44 and aortic valve annulus (not shown) without damaging any leaflets, so that just enough tissue remains to allow sewing of the homograft 12ₐ at the aortic root 50 junction level (not shown). If needed, the chordae tendinea and the corresponding papillary muscle may be further trimmed from the free edge (not shown) of the anterior leaflet 88. Alternatively, the chordae tendinea and the corresponding papillary muscles may be preserved in a standard fashion for attachment to the left ventricular wall (not shown) as disclosed in the '417 patent.

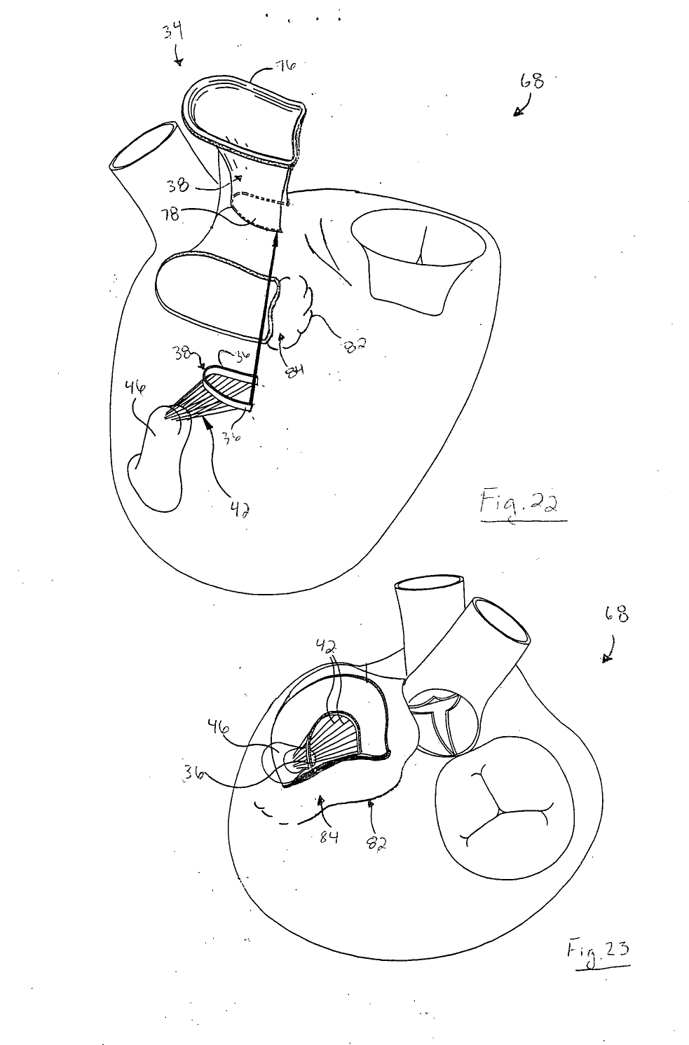

Based on direct or echocardiographic measurements of the native aortic valve 35 (FIG. 20), the native mitral valve 34, the height of the posterior mitral leaflet 40, the chordae tendinae 42 (FIG. 22) and the papillary muscles 46, an appropriately-sized homograft 12ₐ (FIG. 16) is then chosen for implantation into the recipient heart 68. As shown in FIG. 19, an incision 72 is first made to open the left atrium 74 of the recipient heart 68. The incision 72 allows exposure of the native mitral valve 34 and the native aortic valve 35 for excision as shown in FIG. 20.

Next, the native aortic valve 35 and surrounding aortic root 50 are resected as desired. For example, the native aortic root 50 may be resected so that only a subcoronary portion 52 remains (FIG. 21). Alternatively, the native aortic root 50 may be resected so that only a supracoronary portion (not shown) remains.

As illustrated in FIGS. 22 and 23, the native mitral valve 34 is then dissected from the recipient heart 68. A proximal end 76 of the native mitral valve 34 is resected from the mitral valve annulus 82, and a distal end 78 of the native mitral valve, which includes the anterior leaflet 38, is then resected so that the free edge 36 of the anterior leaflet remains intact and connected to the native chordae tendinea 42 which, in turn, remain attached to the corresponding papillary muscles 46. It is important that the rough zone (not shown in detail) and the free edge 36 of the native anterior mitral leaflet 38, the strut chordae tendinea (not shown in detail), and the rough zone chordae tendinea (not shown in detail) are preserved.

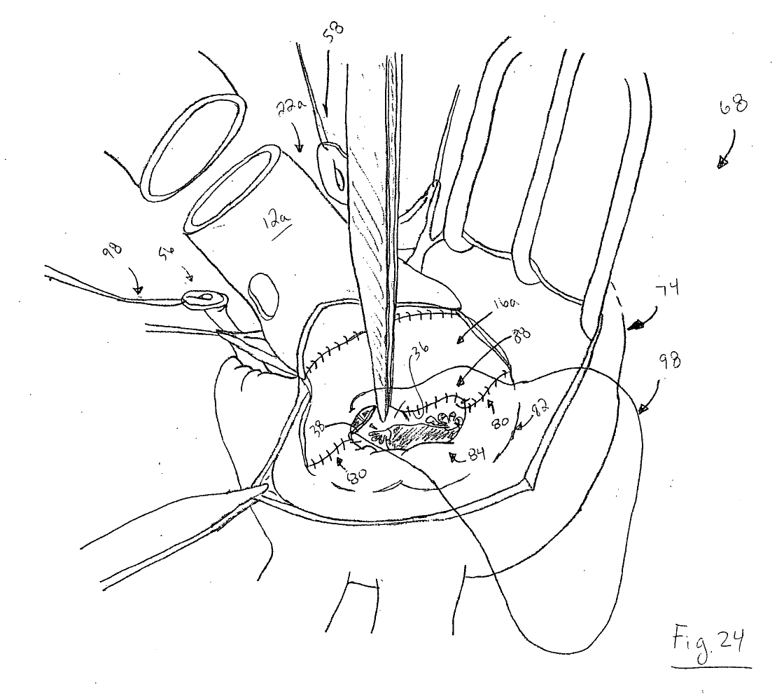

The homograft 12ₐ is next moved into position for implantation. As shown in FIG. 24, the anterior mitral leaflet 88 is sutured down to the free edge 36 of the native anterior mitral leaflet 38 with 5-0 Ethibond or polypropylene (Prolene 5-0) using continuous over-and-over sutures 98. This suture 98 may be started from the apex (or middle) of the anterior mitral leaflet 38 and then directed toward the mitral commissures 80, which makes it easy to suture the anterior mitral leaflet 88 of the homograft 12ₐ to the native anterior leaflet. Additionally, this suture 98 may be continued so that the anterior mitral region 86 of the homograft 12ₐ is joined to the posterior mitral leaflet 40 of the native mitral valve 34 as shown in FIG. 24. During this procedure, the free edge 36 of the native anterior mitral leaflet 38, the rough zone chordae tendinae (not shown), and the cleft chordae tendinae (not shown), except for the basal chordae tendinae (not shown), must be preserved.

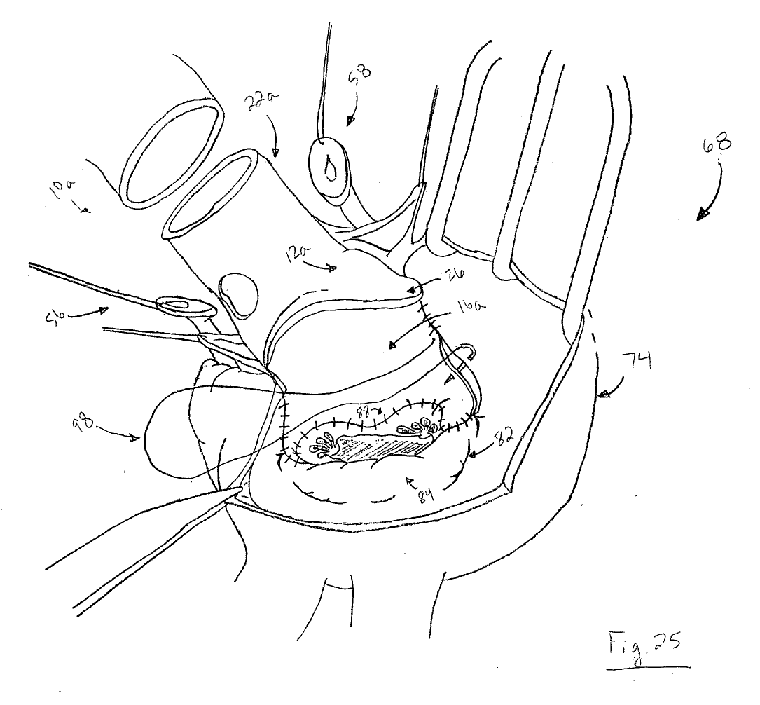

The harvested mitral annulus 44 of the anterior mitral region 86 is then sutured down to the native mitral annulus 82 using continuous or interrupted stitches of 3-0 or 4-0 polypropylene or Ethibond sutures 98 as shown in FIG. 25. The fibrous trigones (not shown) of the homograft 12ₐ are lined up with the fibrous trigones (not shown) of the homograft recipient. Attention is given to distributing the leaflet tissue of the homograft 12ₐ uniformly around the native mitral annulus 82.

Additionally or optionally, the at least a portion 16ₐ of a harvested mitral valve can be supported by remodeling annuloplasty using an appropriately-sized partial or complete annuloplasty mitral ring (not shown). The annuloplasty ring may be secured with sutures placed around the posterior inter-trigonal perimeter of the native mitral annulus 82.

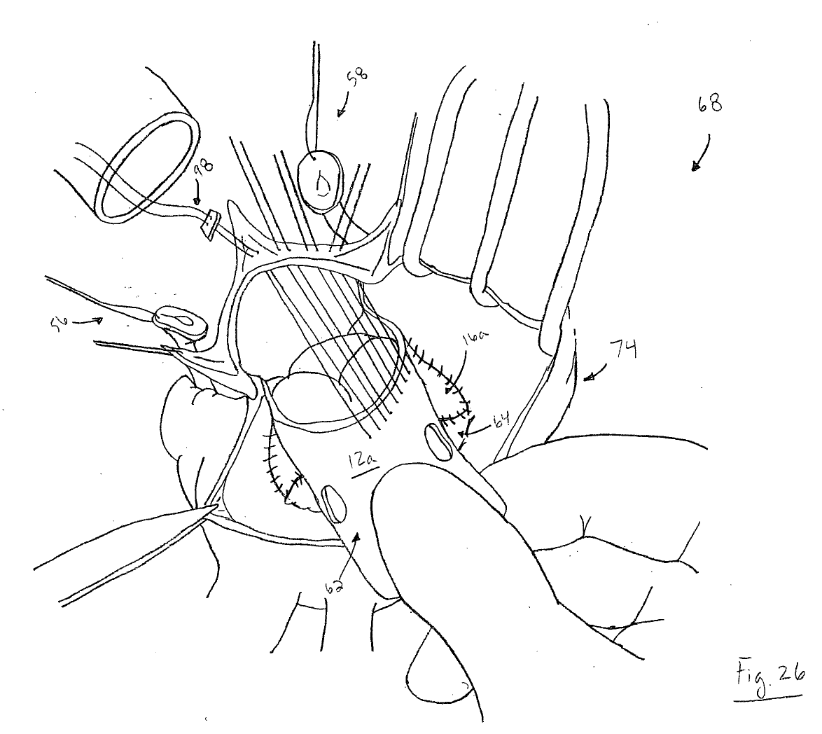

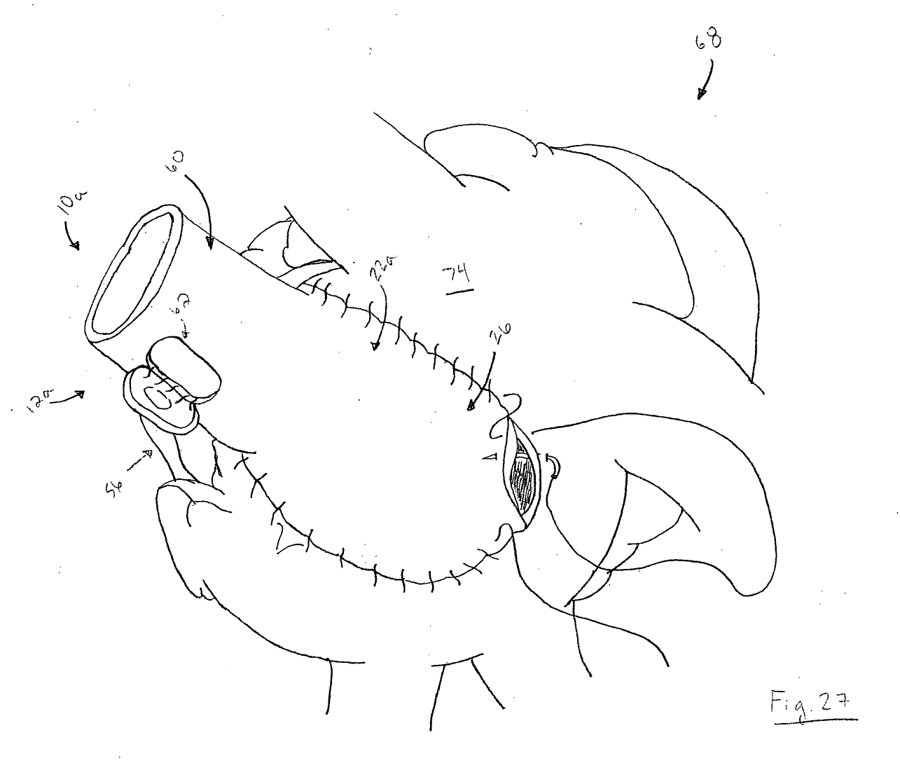

After the at least a portion 16a of a harvested mitral valve is secured in the recipient heart 68, the at least a portion 22ₐ of a harvested distal aorta is then implanted in a known manner using a procedure referred to as a “Mini Root Technique”. As shown in FIGS. 26 and 27, the native right and left coronary sinuses 56 and 58 are attached to the harvested aortic root 24 of the homograft 12ₐ with 4-0 or 5-0 polypropylene or Gore-Tex® sutures 98, and the left atrium 74 is closed using the at least a portion 26 of a harvested atrial wall of the homograft. An exterior view of the recipient heart 68 following implantation of the homograft 12ₐ is shown in FIG. 28.

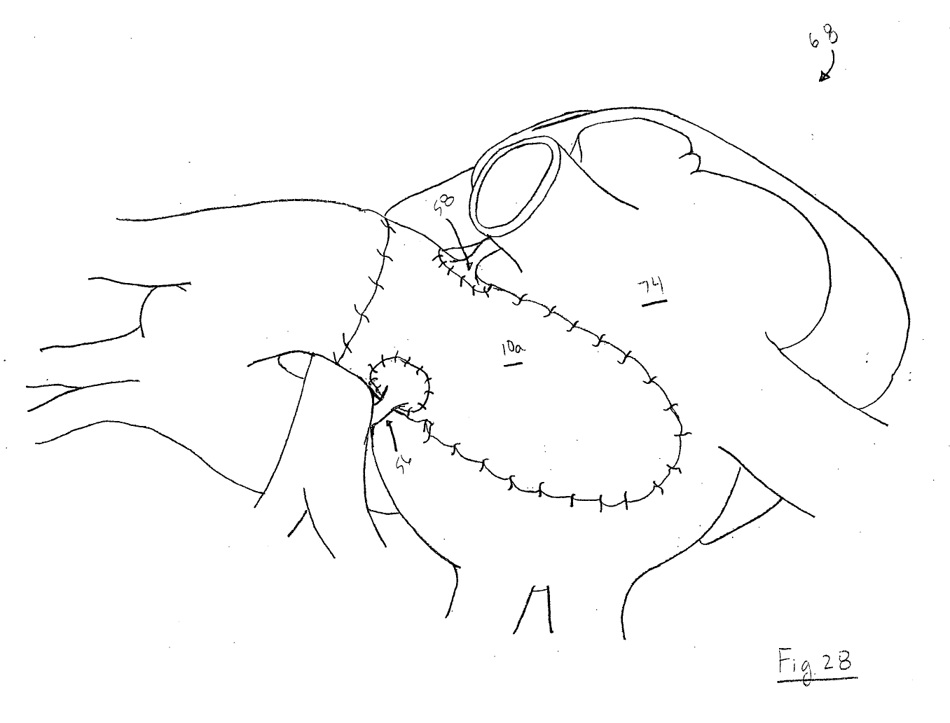

FIG. 29 illustrates an alternative embodiment of the present invention. The bioprosthetic graft 10_{b} of FIG. 29 is identically constructed as the bioprosthetic graft 10 of FIGS. 1-15, except where as described below. In FIG. 29, structures that are identical as structures in FIGS. 1-15 use the same reference numbers, whereas structures that are similar but not identical carry the suffix “b”.

As shown in FIG. 29, the bioprosthetic graft 10_{b} comprises a harvested homograft 12_{b}. The homograft 12_{b} includes a harvested mitral valve portion 16 that can be sutured in place of a native tricuspid valve 100 (FIG. 32). The harvested mitral valve portion 16 (FIG. 29) includes a first leaflet 18 for suturing to the free edges (not shown) of the anterior and posterior native tricuspid valve leaflets 102 and 104, and a second leaflet 20 for suturing to the free edge (not shown) of the septal leaflet 106 of the native tricuspid valve 100. As described in more detail below, the leaflets 102, 104, and 106 of the native tricuspid valve 100 may be left intact following resection of the native tricuspid valve so that the native chordae tendinea 42 continue to provide prolapse prevention and right ventricular muscle support, in addition to maintaining the continuity between the harvested mitral valve annulus 44 and the papillary muscles 46.

Prior to implantation into a recipient heart (not shown), the homograft 12_{b} in FIG. 29 may be harvested as described below. First, the left atrium (not shown) of a donor heart (not shown) is opened. The mitral annulus (not shown), leaflets (not shown), chordae tendinea (not shown) and papillary muscles (not shown) of the donor heart are then anatomically evaluated. The height of the anterior and posterior mitral leaflets is measured by taking either direct or echocardiographic measurements, for example.

Next, the left ventricle (not shown) of the donor heart is opened below the papillary muscle level (not shown). An incision is placed near the donor mitral valve (not shown) and around the posterior area (not shown) of the mitral annulus, taking care to preserve the entire mitral annulus. Doing so ensures that these components of the homograft 12_{b} are intact and remain whole.

The entire mitral valve of the donor heart is then excised or removed by incision of the valve circumferentially. The chordae tendinea that remain attached to the valve leaflets are then removed along with the tips of the corresponding papillary muscles. Alternatively, the chordae tendinea and corresponding tips of the papillary muscles may be preserved. The homograft 12_{b} can then be frozen or otherwise preserved for implantation.

After the homograft 12_{b} is thawed for implantation, any excess myocardium may be trimmed as needed. If needed, the chordae tendinea and the corresponding papillary muscles may be further trimmed from the free edges of the leaflets. Alternatively, the chordae tendinea and the corresponding papillary muscles may be preserved in a standard fashion for attachment to the right ventricular wall using a technique similar to the one disclosed in the '417 patent.

Based on direct or echocardiographic measurements of the native tricuspid valve 100, the height of the native tricuspid leaflets 102, 104, and 106, the chordae tendinea 42 and the corresponding papillary muscles 46, an appropriately-sized homograft 12_{b} is then chosen for implantation into the recipient heart. An incision is first made to open the right atrium of the recipient heart. The incision allows exposure of the native tricuspid valve 100 for excision as shown in FIG. 32.

Next, the native tricuspid valve 100 is dissected from the recipient heart. The native anterior 102, posterior 104, and septal 106 leaflets are dissected from the tricuspid annulus 108 so that the free edges (not shown) of the leaflets remain intact and connected to the native chordae tendinea 42 which, in turn, remain attached to the corresponding papillary muscles 46. The clear zone chordae tendinea (not shown) of the leaflets is then resected. It is important that the rough zone chordae tendinea (not shown) and the strut chordae tendinea (not shown) are preserved.

The homograft 12_{b} is next moved into position for implantation. The free edge (not shown) of the first leaflet 18 of the harvested mitral valve portion 16 of the homograft 12_{b} is sutured down to the native anterior and posterior leaflets 102 and 104 of the native tricuspid valve 100 with 5-0 Ethibond, Gore-Tex®, or polypropylene (Prolene 5-0) continuous over-and-over sutures (not shown). This suture may be started from the apex (or middle) of the anterior and posterior leaflets 102 and 104 and then toward the tricuspid commissures 112 (FIG. 32), which makes it easy to suture the homograft 12_{b} to the native anterior and posterior leaflets. The same procedure is repeated for the attachment of the second leaflet 20 to the native septal leaflet 106. During this procedure for the septal leaflet 106, the rough zone chordae tendinea, the free edge (not shown) of the native septal leaflet, and the cleft chordae tendinea (not shown), except for the basal chordae tendinea (not shown), must be preserved.

The annulus 44 of the harvested mitral valve portion 16 of the homograft 12_{b} is then sutured down to the native tricuspid annulus 108 using continuous or interrupted stitches of 3-0 or 4-0 polypropylene or Ethibond sutures. The fibrous trigones (not shown) of the homograft 12_{b} are lined up with the fibrous trigones (not shown) of the homograft recipient. Attention is given to distributing the leaflet tissue of the homograft 12_{b} uniformly around the native tricuspid annulus 108.

Additionally or optionally, the harvested mitral valve portion 16 of the homograft 12_{b} can be supported by remodeling annuloplasty using an appropriately-sized partial or complete annuloplasty mitral ring (not shown) that is sized to match the septal leaflet 106 of the native tricuspid valve 100, for example. The annuloplasty ring may be secured with sutures placed around the septal inter-trigonal perimeter of the native tricuspid annulus 108.

After the homograft 12_{b} is secured in place of the native tricuspid valve 100, the right atrium is then closed by suturing the incision. The homograft 12_{b} may then serve as a normally functioning cardiac valve.

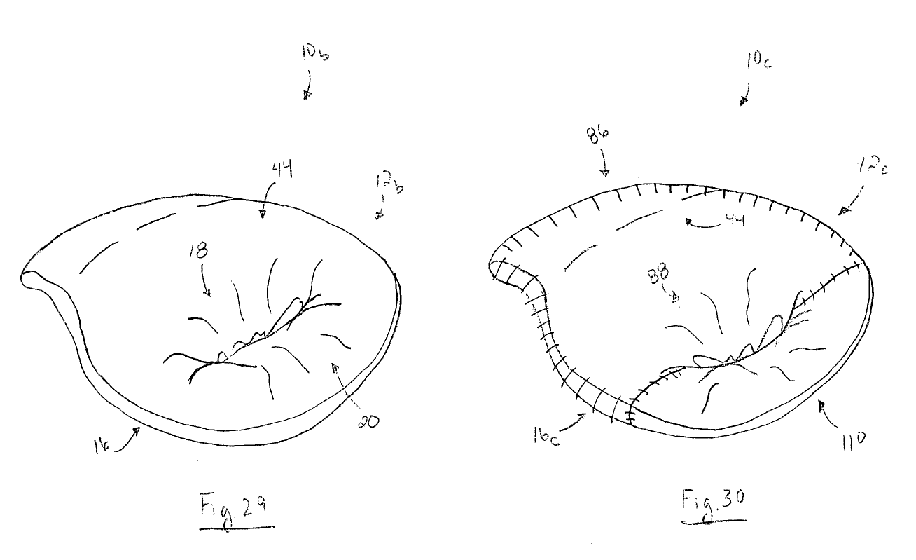

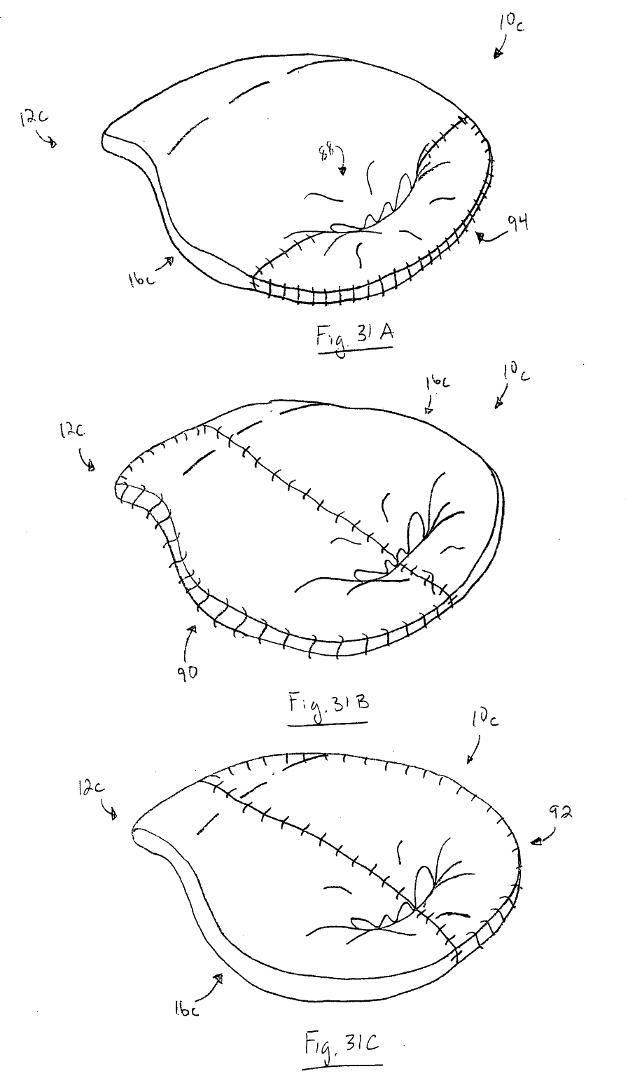

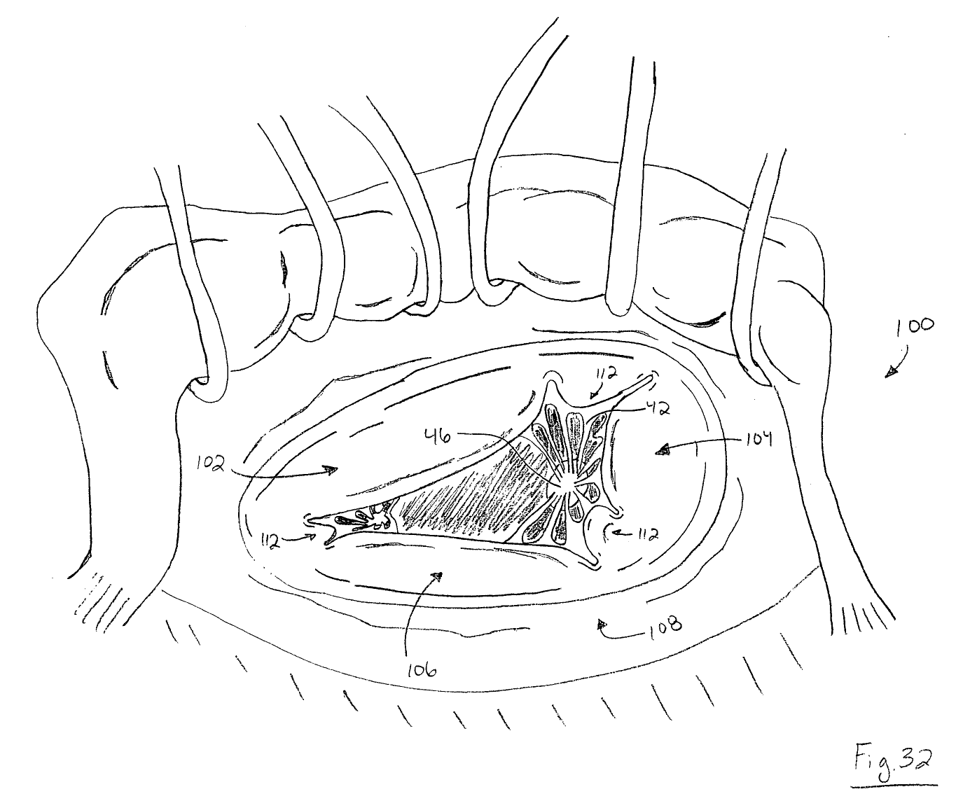

FIGS. 30-31 illustrate an alternative embodiment of the present invention. The bioprosthetic graft 10_{c} of FIGS. 30-31 is identically constructed as the bioprosthetic graft 10ₐ of FIGS. 16-28, except where as described below. In FIGS. 30-31, structures that are identical as structures in FIGS. 16-28 use the same reference numbers, whereas structures that are similar but not identical carry the suffix “c”.

As shown in FIG. 30, the bioprosthetic graft 10_{c} comprises a harvested homograft 12_{c}. The homograft 12_{c} includes at least a portion 16_{c} of a harvested mitral valve that can be sutured to a partial section 110 of a native tricuspid valve 100 (FIG. 32), such as the septal leaflet 106 of the tricuspid valve. The at least a portion 16_{c} (FIG. 30) of a harvested mitral valve can include an anterior mitral region 86 comprising an anterior leaflet 88 as shown in FIG. 30. Alternatively, the at least a portion 16_{c} of a harvested mitral valve can include a posterior mitral region 94 (FIG. 31 A), anterolateral portion 90 (FIG. 31 B), or a posteromedial portion 92 (FIG. 31C).

Prior to implantation, the homograft 12_{c} shown in FIG. 30 may be harvested as described below. First, the left atrium (not shown) of a donor heart (not shown) is opened and the mitral annulus (not shown), posterior mitral leaflet (not shown), chordae tendinea (not shown) and papillary muscles (not shown) of the donor heart are then anatomically evaluated. Next, the height of the posterior mitral valve leaflet is measured by taking either direct or echocardiographic measurements, for example.

Next, the left ventricle (not shown) of the donor heart is opened below the papillary muscle level (not shown). An incision is placed near the donor mitral valve (not shown) and around the posterior area (not shown) of the mitral annulus, taking care to preserve the mitral valve annulus. Doing so ensures that these components of the homograft 12_{c} remain intact and are preserved as a whole.

A portion of the native tricuspid valve 100 of a recipient heart (not shown) is then excised or removed by incision. Where, for example, the native anterior and posterior leaflets 102 and 104 are diseased, the donor mitral valve may be excised so that the at least a portion 1 6_{c} of a harvested mitral valve only includes the anterior leaflet 88 and surrounding mitral annulus 44. The chordae tendinea that remain attached to the anterior mitral valve leaflet 88 are then removed along with the tip of the corresponding papillary muscles. Alternatively, the chordae tendinea and corresponding tip of the papillary muscles may be preserved. The homograft 12_{c} can then be frozen or otherwise preserved for implantation.

After the homograft 12_{c} is thawed for implantation, any excess myocardium may be trimmed as needed. If needed, the chordae tendinea and the corresponding papillary muscles may be further trimmed from the free edge (not shown) of the anterior mitral leaflet 88. Alternatively, the chordae tendinea and the corresponding papillary muscles may be preserved in a standard fashion for attachment to the right ventricular wall (not shown) in a manner similar to the one described in the '417 patent.

Based on direct or echocardiographic measurements of the native tricuspid valve 100, the height of the anterior and posterior tricuspid leaflets 102 and 104, the native chordae tendinae 42, and the native corresponding papillary muscles 46, an appropriately-sized homograft 12_{c} is then chosen for implantation into the recipient heart. An incision is first made to open the right atrium (not shown) of the recipient heart. The incision allows exposure of the native tricuspid valve 100 as shown in FIG. 32.

Next, the native tricuspid valve 100 is dissected from the recipient heart. The native anterior and posterior leaflets 102 and 104 are dissected from the tricuspid annulus 108 so that the free edges (not shown) of the leaflets remain intact and connected to the native chordae tendinea 42 which, in turn, remain attached to the corresponding papillary muscles 46. The clear zone chordae tendinea (not shown) of both leaflets 102 and 104 is then resected. It is important that the rough zone chordae tendinea (not shown) and the strut chordae tendinea (not shown) are preserved.

The homograft 12_{c} is next moved into position for implantation. The free edge (not shown) of the anterior leaflet 88 of the homograft 12_{c} is sutured down to the native anterior and posteriori leaflets 102 and 104 of the native tricuspid valve 100 with 5-0 Ethibond, Gore-Tex®, or polypropylene (Prolene 5-0) continuous over-and-over sutures (not shown). During this procedure, the rough zone chordae tendinea, the free edges (not shown) of the native anterior and posterior leaflets 102 and 104, and the cleft chordae tendinea (not shown), except for the basal chordae tendinea (not shown), must be preserved.

The annulus 44 of the at least a portion 16_{c} of a harvested mitral valve is then sutured down to the native tricuspid annulus 108 using continuous or interrupted stitches of 3-0 or 4-0 polypropylene or Ethibond sutures. The fibrous trigones (not shown) of the homograft 12_{c} are lined up with the fibrous trigones (not shown) of the homograft recipient. Attention is given to distributing the leaflet tissue of the homograft 12_{c} uniformly around the native tricuspid annulus 108.

Additionally or optionally, the at least a portion 16_{c} of a harvested mitral valve of the homograft 12_{c} can be supported by remodeling annuloplasty using an appropriately-sized partial or complete annuloplasty mitral ring (not shown) that is sized to match the septal leaflet 106 of the native tricuspid valve 100, for example. The annuloplasty ring may be secured with sutures placed around the septal inter-trigonal perimeter of the native tricuspid annulus 108.

After the homograft 12_{c} is secured in place of the native tricuspid valve 100, the right atrium is then closed by suturing the incision. The homograft 12_{c} may then serve as a normally functioning cardiac valve.

From the above description of the invention, those skilled in the art will perceive improvements, changes and modifications. For example, the present invention may be implanted using the standard mitral valve homograft-papillary muscle technique disclosed in the '417 patent. Such improvements, changes and modifications within the skill of the art are intended to be covered by the appended claims.

Having described the invention, I claim:

1. A stentless bioprosthetic graft for repairing both a first native heart valve and a second native heart valve of a heart, said bioprosthetic graft comprising:
a harvested homograft that includes a harvested mitral valve portion and a harvested distal aorta, said harvested distal aorta including a harvested aortic root, a harvested aortic valve, and at least a portion of a harvested atrial wall, said harvested mitral valve portion for suturing in place of the first native heart valve, said distal aorta for suturing to a partial section of the second native heart valve; and
an extension portion made of a biocompatible material, said extension portion being sutured to said homograft and for suturing to the left atrial wall of the heart to close an incision in the left atrial wall following implantation of said harvested mitral valve portion and said harvested distal aorta of said homograft.

2. The bioprosthetic graft of claim 1, wherein said mitral valve portion of said homograft includes first and second leaflets for suturing to free edges of the anterior and posterior leaflets of the native mitral valve that are left intact following resection of the native mitral valve so that the native chordae tendinea continue to provide prolapse prevention and left ventricular muscle support in addition to maintaining the continuity between the valve annulus and the papillary muscles.

3. The bioprosthetic graft of claim 1, wherein said partial section of the second native heart valve comprises a supracoronary portion of the native aortic root.

4. The bioprosthetic graft of claim 1, wherein said partial section of the second native heart valve comprises a subcoronary portion of the native aortic root.

5. A method for repairing a first native heart valve and a second native heart valve of a heart with a bioprosthetic graft, said method comprising the steps of:
harvesting a homograft that includes both a harvested mitral valve portion, a harvested distal aorta, and a biocompatible extension portion, the distal aorta including a harvested aortic root, a harvested aortic valve, and at least a portion of a harvested atrial wall, the harvested mitral valve portion for suturing in place of the first native heart valve, the harvested distal aorta for suturing to a partial section of the second native heart valve;
resecting the majority of the anterior and posterior leaflets of the first native heart valve from the valve annulus but leaving the free edges of the anterior and posterior leaflets intact along with the native chordae tendinea so that the native chordae tendinea can provide prolapse prevention and left ventricular muscle support for the harvested mitral valve portion of the homograft in addition to maintaining the continuity between the valve annulus and the papillary muscles;
suturing the biocompatible extension portion to the homograft;
suturing first and second leaflets of the harvested mitral valve portion of the homograft to the free edges of the anterior and posterior leaflets of the first native heart valve that remain following resection of the first native heart valve;
suturing the annulus of the harvested mitral valve portion of the homograft to the annulus of the first native heart valve to secure the bioprosthetic graft to the valve annulus;
suturing the harvested distal aorta of the homograft to the partial section of the second native heart valve; and
suturing the extension portion to the left atrial wall of the heart to close the left atrial wall.

6. A stentless bioprosthetic graft for repairing both a first native heart valve and a second native heart valve, said bioprosthetic graft comprising:
a harvested homograft that includes at least a portion of a harvested mitral valve and at least a portion of a harvested distal aorta, said harvested distal aorta including a harvested aortic root, a harvested aortic valve and at least a portion of a harvested atrial wall, said at least a portion of a harvested mitral valve for suturing to a partial section of the first native heart valve, said at least a portion of a harvested distal aorta for suturing to a partial section of the second native heart valve, said at least a portion of a harvested atrial wall for suturing to the native atrial wall of the heart to close the native atrial wall following implantation of the harvested homograft.

7. The bioprosthetic graft of claim 6, wherein said at least a portion of a harvested mitral valve comprises a posterior leaflet.

8. The bioprosthetic graft of claim 6, wherein said at least a portion of a harvested mitral valve comprises an anterior leaflet.

9. The bioprosthetic graft of claim 6, wherein said at least a portion of a harvested mitral valve comprises an anterolateral portion.

10. The bioprosthetic graft of claim 6, wherein said at least a portion of a harvested mitral valve comprises a posteromedial portion.

11. The bioprosthetic graft of claim 6, wherein said partial section of the second native heart valve comprises a supracoronary portion of the native aortic root.

12. The bioprosthetic graft of claim 6, wherein said partial section of the second native heart valve comprises a subcoronary portion of the native aortic root.

13. A method for repairing both a first native heart valve and a second native heart valve of a heart with a bioprosthetic graft, the first native heart valve having at least one leaflet connected to an annulus, the at least one leaflet having a free edge and being further connected to papillary muscles by chordae tendinea, said method comprising the steps of:
harvesting a homograft that includes at least a portion of a harvested mitral valve and at least a portion of a harvested distal aorta, the distal aorta including a harvested aortic root, a harvested aortic valve and at least a portion of a harvested atrial wall, the at least a portion of a harvested mitral valve for suturing to a partial section of the first native heart valve, and the at least a portion of a harvested distal aorta for suturing to a partial section of the second native heart valve;
resecting at least a portion of the first and second native heart valves from the heart;
suturing the at least a portion of a harvested mitral valve in place of the first native heart valve;
suturing the at least a portion of a harvested distal aorta in place of the second native heart valve; and
suturing the at least a portion of a harvested atrial wall to the native atrial wall of the heart to close the native atrial wall.

14. The method of claim 13, wherein said step of suturing the at least a portion of a harvested mitral valve in place of the first native heart valve further comprises the steps of:
resecting the majority of the at least one leaflet of the first native heart valve from the valve annulus but leaving the free edge of the at least one leaflet intact along with the native chordae tendinea so that the native chordae tendinea can provide prolapse prevention and left ventricular muscle support for the at least a portion of a harvested mitral valve in addition to maintaining the continuity between the valve annulus and the papillary muscles;
suturing a portion of the at least a portion of a harvested mitral valve to the free edge of the at least one leaflet of the first native heart valve that remain following resection of the first native heart valve; and
suturing the at least a portion of a harvested mitral valve to the valve annulus of the first native heart valve to secure the at least a portion of a harvested mitral valve to the valve annulus.

15. The method of claim 13, wherein said step of resecting the native first and second heart valves from the heart further comprises the step of resecting a supracoronary portion of the native aortic root.

16. The method of claim 13, wherein said step of resecting the native first and second heart valves from the heart further comprises the step of resecting a subcoronary portion of the native aortic root.

17. The method of claim 13, wherein said step of suturing the at least a portion of a harvested distal aorta in place of the second native heart valve further comprises the step of suturing the supracoronary portion of the native aortic root to the at least a portion of a harvested distal aorta

18. A stentless bioprosthetic graft for repairing a native tricuspid valve of a heart, said bioprosthetic graft comprising:
a harvested homograft that includes at least a portion of a harvested mitral valve, said at least a portion of a harvested mitral valve for suturing to a partial section of the native tricuspid valve.

19. The bioprosthetic graft of claim 18, wherein said partial section of the native tricuspid valve includes a septal leaflet of the native tricuspid valve.

20. The bioprosthetic graft of claim 18, wherein said at least a portion of a harvested mitral valve comprises a posterior leaflet.

21. The bioprosthetic graft of claim 18, wherein said at least a portion of a harvested mitral valve comprises an anterior leaflet.

22. The bioprosthetic graft of claim 18, wherein said at least a portion of a harvested mitral valve comprises an anterolateral portion.

23. The bioprosthetic graft of claim 18, wherein said at least a portion of a harvested mitral valve comprises a posteromedial portion.

24. A method for repairing a native tricuspid valve of a heart with a bioprosthetic graft, the native tricuspid valve having at least one leaflet connected to an annulus, the at least one leaflet having a free edge and being further connected to papillary muscles by chordae tendinea, said method comprising the steps of:
harvesting a homograft that includes at least a portion of a harvested mitral valve, the at least a portion of a harvested mitral valve for suturing to a partial section of the native tricuspid valve;
resecting at least a portion of the native tricuspid valve from the heart; and
suturing the at least a portion of a harvested mitral valve to the partial section of the native tricuspid valve.

25. The method of claim 24, wherein said step of suturing the at least a portion of a harvested mitral valve in place of the native tricuspid valve further comprises the steps of:
resecting the majority of the at least one leaflet of the native tricuspid valve from the valve annulus but leaving the free edge of the at least one leaflet intact along with the native chordae tendinea so that the native chordae tendinea can provide prolapse prevention and right ventricular muscle support for the at least a portion of a harvested mitral valve in addition to maintaining the continuity between the native valve annulus and the papillary muscles;
suturing a portion of the at least a portion of a harvested mitral valve to the free edge of the at least one leaflet of the native tricuspid valve that remains following resection of the native tricuspid valve; and
suturing at least a portion of a harvested mitral valve to the valve annulus of the native tricuspid valve to secure the at least a portion of a harvested mitral valve to the valve annulus.

26. A stentless bioprosthetic graft for repairing a native tricuspid valve of a heart, said bioprosthetic graft comprising:
a harvested homograft that includes a harvested mitral valve, said harvested mitral valve for suturing in place of the native tricuspid valve.

27. The bioprosthetic graft of claim 26, wherein said harvested mitral valve of said homograft includes at least one leaflet for suturing to at least one free edge of at least one leaflet of the native tricuspid valve that is left intact following resection of the native tricuspid valve so that the native chordae tendinea continue to provide prolapse prevention and right ventricular muscle support in addition to maintaining the continuity between the native valve annulus and the papillary muscles.

28. A method for repairing a native tricuspid valve of a heart with a bioprosthetic graft, the native tricuspid valve having at least one leaflet connected to an annulus, the at least one leaflet having a free edge and being further connected to papillary muscles by chordae tendinea, said method comprising the steps of:
harvesting a homograft that includes a harvested mitral valve, the harvested mitral valve for suturing in place of the native tricuspid valve;
resecting the native tricuspid valve from the heart; and
suturing the harvested mitral valve in place of the native tricuspid valve.

29. The method of claim 28, wherein said step of suturing the harvested mitral valve in place of the native tricuspid valve further comprises the steps of:
resecting the majority of the at least one leaflet of the native tricuspid valve from the valve annulus but leaving the free edge of the at least one leaflet intact along with the native chordae tendinea so that the native chordae tendinea can provide prolapse prevention and right ventricular muscle support for the harvested mitral valve in addition to maintaining the continuity between the native valve annulus and the papillary muscles;
suturing a portion of the harvested mitral valve to the free edge of the at least one leaflet of the native tricuspid valve that remains following resection of the native tricuspid valve; and
suturing a portion of the harvested mitral valve to the valve annulus of the native tricuspid valve to secure the harvested mitral valve to the valve annulus.
